(19) United States
(12) Patent Application Publication
(10) Pub. No.: US 2025/0169504 A1
(43) Pub. Date: May 29, 2025

(54) MULTI-TARGET BIOINSECTICIDAL AGRICULTURAL COMPOSITION COMPRISING BACTERIA AND THEIR METABOLITES APPLIED TO CROPS OF AGRICULTURAL IMPORTANCE

(71) Applicant: TOTAL BIOTECNOLOGIA INDÚSTRIA E COMÉRCIO S/A, Curitiba - PR (BR)

(72) Inventors: Josiane Fukami, Curitiba - PR (BR); Douglas Fabiano Gomes, Curitiba - PR (BR); Juliana Marcolina Gomes, Curitiba - PR (BR); Jonas Hipolito de Assis Filho, Curitiba - PR (BR)

(21) Appl. No.: 18/843,110

(22) PCT Filed: Mar. 9, 2022
(86) PCT No.: PCT/BR2022/050080

§ 371 Date: Aug. 30, 2024

(51) Int. Cl. A01N 63/27; A01N 63/20; A01P 7/04; C12N 1/20

(52) U.S. Cl. CPC A01N 63/27; A01N 63/20; A01P 7/04; C12N 1/20

(57) ABSTRACT

The present invention relates to an agricultural composition containing one or more species of the genus Pseudomonas and the genus Chromobacterium and the process of industrially inducing their metabolites to increase the multi-target bioinsecticidal efficiency of microorganisms in the field application to plants of agricultural interest. Surprisingly, the agricultural composition with one or more species of Pseudomonas or the mixture with Chromobacterium with different doses of application can act on several pests and crops of agronomic interest, such as corn, soybean and bean. Finally, the agricultural composition alternatively promotes plant growth by inducing metabolites such as phytohormone AIA and the production of siderophores, which are capable of improving plant health.

### FIELD OF THE INVENTION

### Technical Field

The present invention relates to a multi-target bioinsecticidal agricultural composition comprising at least one bacterial species from the genus Pseudomonas or the genus Chromobacterium, or a mixture thereof, wherein the composition is enriched in metabolites produced by said bacterial species. The present invention further involves a process for producing said agricultural composition, controlling diseases and crop pests, plants of agricultural interest and promoting plant growth.

### Description of the Related Art

In recent years, crop intensification and increase associated with the excessive and successive use of insecticide molecules having similar mechanisms of action has caused undesirable selections in pest insect populations of agricultural interest, resulting in increased resistance of these populations to traditional solutions (agrochemicals). As a result of this process, spraying of agrochemicals becomes more frequent and voluminous, contrary to a healthier and more rational agriculture. For that reason and in line with the consumers' concern on food quality, studies on new pest insect control and management tools have been intensified (TAVARES et al., 2009). Also, biological control using microorganisms, such as fungi and bacteria, is an important tool for the management of resistance and the conscious use of agrochemicals. Of notorious knowledge are bacteria from the genus Bacillus, in particular, B. thuringiensis, which since 1915 has been shown to have potential insecticidal activity against insects of the order of Lepidoptera (Habbib and Andrade, 1986). In 1938 commercialization of such bioinsecticidal product was started in France (Daust, 1990), which was later spread throughout the world.

Since then, efforts have been intensified in the search for new bacteria genera exhibiting bioinsecticidal activity, which in resulted the identification of Pseudomonas and Chromobacterium bacteria with potential bioinsecticidal activity. In particular, Pseudomonas fluorescens has been widely studied due to its action against several insect orders (Dangar, 2008; Kupferschmied et al., 2013; Suganthi et al. 2017). In the case of Pseudomonas greater evidence of insecticidal chlororaphis species, action was observed for insects belonging to the order Lepidoptera (Anderson and Kim, 2018). Bacteria from the Chromobacterium genus have also been studied and there are reports that they were shown to have insecticidal potential against insects of the orders Coleoptera and Hemiptera (Martin et al., 2007).

The reported insecticidal function in Pseudomonas is directly related to the synthesis of specific metabolites, including hydrogen cyanide (HCN) and pyrrolnitrine (Schippers et al., 1990; Lee et al., 2010). In addition, enzymes such as proteases, phospholipase C and chitinases, as well as the production of FitD toxin, also contribute to the insecticidal activity carried out by bacteria of this genus (Martin et al., 20007; Flury et al., 2017). Or even the production of violacein pigment by some Chromobacterium species having an antimicrobial potential and some studies with insecticidal potential (Duran et al., 1983; 1994; Martin et al., 20007). Induction of some of these metabolites can take place in the presence of specific amino acids, such as glycine and tryptophan, which are HCN and pyrrolenitrine precursors, respectively (Anderson and Kim, 2018; Lee et al., 2010).

Another major strategy for pest control, including insects of the orders Lepidoptera, Diptera, Hemiptera, among others, uses natural plant extracts. Among these extracts basil and citronella essential oils have been reported as natural insecticides. In addition to these, Neem extract, which is extracted from plants belonging to the genus Azadirachta, has azadirachtin as an active ingredient and is described to have pronounced insecticidal activity. Although well known, association of this compound with microorganisms in insecticidal compositions is challenging, being limited due to incompatibility, as they also have an antimicrobial effect.

In addition to the bioinsecticidal activity, the use of beneficial microorganisms in agriculture has been increasingly frequent to seek different aspects such as growth promotion. The term “plant growth-promoting bacteria (PGPB)” was first used by Klopper and Schroth (1978) to describe soil bacteria that colonize the roots and/or rhizosphere of plants s and enhance their growth. These bacteria have been extensively studied in recent years, generating important results on the mechanisms they play and providing better plant development. Among the different genera of microorganisms characterized as PGPB are agrobacterium, Allorhizobium, Arthrobacter, Azospirillum, Azotobacter, Bacillus, Bradyrhizobium, Burkholderia, Caulobacter, Chromobacterium, Erwinia, Exiguobacterium, Flavobacterium, Mesorhizobium, Micrococcous, Providencia, Pseudomonas, Rhizobium and Serratia (Yadav et al., 2017; Suman et al., 2015; Suman et al., 2016).

Differentiation of microorganisms as plant growth promoters is linked to the identification of one or more mechanisms of action, with emphasis on the solubilization of phosphorus (Pikovskaya, 1948), zinc (Fasim et al., 2002) and potassium (Hu and Guo, 2006), the production of phytohormones such as auxins (Bric et al., 1991) and gibberellins (Brown, 1968), biological nitrogen fixation (Boddey et al., 1995) and the production of ACC-deaminase enzyme (Jacobson et al., 1994). Furthermore, the biosynthesis of ammonia (Cappucino and Sherman, 1992) and siderophores (Schwyn and Neilands, 1987) are further attributes of bacteria that benefit plants. In addition to the already mentioned mechanisms of action, it is worth mentioning the production of siderophores, a compound produced by microorganisms that is associated with plant growth promotion, that is, either directly by facilitating the acquisition of iron (Fe), or indirectly, by inhibiting the establishment of pathogens by sequestering Fe from the environment, thus limiting the amount of this metal available for pathogen growth, hence providing better plant health (RADZKI et al., 2013). Another important feature of growth-promoting microorganisms is the biosynthesis of plant hormones (phytohormones), such as indoleacetic acid (IAA), an important auxin for the growth of apical buds that acts directly on the growth of roots and shoots of plants.

However, there is still a need for biotechnological solutions for effective biological control of insect pests in crops of agricultural interest, mainly species belonging to the order Hemiptera, contributing to the reduction of the load of chemical pesticides applied in the environment and, therefore, improving with the agribusiness sustainability.

### BRIEF DESCRIPTION OF THE INVENTION

The present invention provides a multi-target bioinsecticidal agricultural composition surprisingly effective in the biological control of insect pests in plant crops of agricultural interest, including species belonging to the order Hemiptera.

The agricultural composition according to the present invention comprises at least one bacterial species of the genus Pseudomonas or the genus Chromobacterium, or a mixture thereof, wherein the composition is enriched in metabolites produced by said bacterial species.

An agricultural composition according to the present invention is especially useful in the control of insect pests of agricultural interest, and can be used in different crops and product concentrations, as well as at different times of application. In a preferred embodiment, a composition according to the present invention is effective in controlling insect pests belonging to the order Hemiptera.

The present invention further provides a process for producing an agricultural composition enriched in bacterial metabolites according to the present invention by means of culture parameters that induce the production of said bacterial metabolites.

The present invention further provides a process of controlling diseases and crop pests of plants of agricultural interest comprising the application of a composition according to the present invention to agricultural crops.

The present invention further provides a process of controlling resistance of insect pests against agrochemicals in crops of plants of agronomic interest comprising applying a composition according to the present invention to the agricultural crop.

In another embodiment, the present invention provides a method of promoting the growth of crop plants comprising applying an agricultural composition according to the present invention to the agricultural crop.

### BRIEF DESCRIPTION OF THE FIGURES/DRAWINGS

For a full understanding of the invention, the following figures should be construed as illustrative only and not limitative in any way of the scope of the invention.

FIG. 1 illustrates the natural infestation of adult leafhoppers (Dalbulus maidis) 25 days after three applications of formulations of Pseudomonas species with no induction of metabolite production or with induction of the metabolites according to the present invention at different doses of application in corn crop. * Results from 8 treatments and 4 replicates, data not presented as they are not relevant to the study. Scott&Knott test (p≤0.1) with a randomized complete block (RCB) experimental design.

FIG. 2 illustrates the percentage of incidence of corn stunt disease 25 days after three applications of formulations of Pseudomonas species with no induction of metabolite production or with induction of metabolites according to the present invention at different doses of application in corn crop. * Results from 8 treatments and 4 replicates, data not presented for being irrelevant to the study. Means followed by the same letters do not differ from each other by Tukey's test at 5% significance.

FIG. 3 illustrates the percentage of mortality of whitefly (Bemisia tabaci) nymphs after application of the formulation of Pseudomonas, P. chlororaphis and P. fluorescens species, with no induction of the production of metabolites or with induction of the metabolites according to the present invention at different doses to bean crop. *Results of 10 treatments and 4 replications, data not presented for being irrelevant to the he study Scott&Knott Test (p≤0.0.05) with a randomized complete block design (RCB).

FIG. 4 illustrates the percentage of brown bug (Euschistus heros) mortality in a bioassay after application of the formulation comprising a mixture of Pseudomonas and Chromobacterium species at different concentrations of the inducer of metabolites according to the present invention. * Results from 9 treatments and 4 replicates, data not presented for being irrelevant to the study. Scott&Knott test (p≤0.0.05) with a completely randomized design.

FIG. 5 illustrates the natural infestation of brown stink bug nymphs and adults 26 days after the three applications of the formulations with induction of the metabolites of Pseudomonas and Chromobacterium species and the mixture of Pseudomonas and Chromobacterium species at different application doses in soybean crop. Scott&Knott test (p≤0.05) with a randomized complete block (RCB) experimental design. Caption: DAA: Days After Application. The first number denotes the first or second application. The second number denotes how many days after the first or second assessment the assessment was performed. For example, DAA1:3 means assessment performed 3 days after the first application; DAA2:6 means assessment performed 6 days after the second application.

FIG. 6 illustrates the concentration of AIA synthesized by with different Pseudomonas species concentrations of secondary metabolite-inducing molecules and the same concentration of tryptophan precursor. Formulations 1, 2 and 3 showed higher, intermediate and lower concentrations, respectively, of the secondary metabolite inducing molecule. Formulation 1: Change in glycine concentration from 1 to 2 g/L; Formulation 2: Glycine change of from 2.1 to 5 g/L and Formulation 3: glycine change of from 5.1-10 g/L.

FIG. 7 illustrates the percentage of siderophore production synthesized by Pseudomonas species with different concentrations of secondary metabolite-inducing molecules and the same concentration of tryptophan precursor. Formulations 1, 2 and 3 showed higher, intermediate and lower concentrations, respectively, of the secondary metabolite inducing molecule. Formulation 1: Change in glycine concentration from 1 to 2 g/L; Formulation 2: Glycine change of from 2.1 to 5 g/L and Formulation 3: glycine change of from 5.1-10 g/L.

### DETAILED DESCRIPTION OF THE INVENTION

The present invention provides a multi-target bioinsecticidal agricultural composition comprising at least one bacterial species of the genus Pseudomonas or the genus Chromobacterium, or mixture a thereof, wherein the composition is enriched in metabolites produced by said bacterial species.

Pseudomonas bacterial species that can be used in accordance with the present invention include but are not limited to Pseudomonas chlororaphis (Anderson and Kim, 2018), P. fluorescens (Dangar, 2008; Kupferschmied et al., 2013; Suganthi et al. 2017) P. protens, P. tamsuii and P. taiwanensis. Bacterial species from the genus Chromobacterium that can be used in accordance with the present invention include Chromobacterium subtsugae (Duran et al., 1983; 1994; Martin et al., 20007), C. violaceum, C. sphagni, C. vaccinii e C. piscinae.

In a preferred embodiment of the present invention, the agricultural composition comprises a mixture of species from both genera (Pseudomonas and Chromobacterium).

In one embodiment of the present invention, the ratio of each microorganism in the composition is from about 60% to about 80% Pseudomonas, from about 20% to about 40% Chromobacterium. In a preferred embodiment, the ratio of each microorganism in the composition is from about 40% to about 60% (v/v) Pseudomonas chlororaphis, from about 10% to about 20% (v/v) P. fluorescens and from about 20% to about 50% (v/v) Chromobacterium subtsugae.

In a preferred embodiment of the present invention the bacterial metabolites include, for example, indole acetic acid (IAA), HCN, pyrronitrine, FitD toxins, violacein pigment and siderophores.

The present invention further provides an industrial process of producing an agricultural composition according to the present invention involving parameters that promote high cell concentration during the exponential phase of bacterial growth, induction of the biosynthesis of bacterial metabolites in the stationary phase, as well as stability of said metabolites and cell viability for application as an agricultural composition.

The process for producing an agricultural composition according to the present invention comprises the steps of:
(a) fermenting at least one bacterial culture comprising at least one bacterial species of the genus Pseudomonas or the genus Chromobacterium, or a mixture thereof, to obtain a fermented broth;
(b) inducing the production of metabolites by the bacteria present in the fermented broth upon cultivation in a medium comprising glycine and/or tryptophan; and
(c) formulating an agricultural composition comprising the fermented broth and, optionally, additional agriculturally acceptable components.

Preferred process parameters such as growth temperature, air volume, stirring and pressure (related to the rate of dissolved oxygen in the culture medium) are given below.

In a preferred embodiment according to the present invention, fermentation (step (a)) of the bacterial culture containing one or more bacterial species according to the present invention per batch takes place for about 24 to about 168 hours.

In a preferred embodiment, the process of the present invention comprises the sequential expansion (up scaling) of the bacterial culture for inoculation of the fermentation culture. Preferably, the sequential expansion is started on a culture with a volume of about 100 mL, which serves as an inoculum for cultivation in a volume of about 1 L. Next, the culture of about 1 L is inoculated in a volume of approximately 10 L, which, in turn, is inoculated into two flasks in tanks of approximately 180 L and which are finally transferred to reactors of about 2,000 L.

In a preferred embodiment, the bacterial culture is expanded in flasks of about 100 mL by incubation on an orbital shaker at about 80 rpm at about 200 rpm. Incubation time is preferably from about 8 hours to about 48 hours. Preferably, the bacterial culture is grown in culture medium flasks of around 1 L by incubation on an orbital shaker at 80 rpm to 200 rpm.

In a preferred embodiment, the air flow rate of about 10 L stainless steel flasks is, preferably, from about 0.25 to about 1.5 Nm3/h (=0.41−2.5 vvm) and the incubation time is preferably about 8 hours to about 48 hours.

In a preferred embodiment, the incubation temperature for expansion of the bacterial culture cells according to the present invention is about 22° C. to about 38° C.

In a preferred embodiment where the agricultural composition according to the present invention comprises more than one Pseudomonas and/or Chromobacterium species the bacterial cultures are grown separately in the up scaling process to about 180 L and mixed in about 2000 L fermenters. To this end, in a preferred embodiment, after cultivation of Pseudomonas and Chromobacterium in flasks of about 1 L culture medium, they are inoculated into two stainless steel flasks containing about 10 L of culture medium, the culture medium being up scaled to about 10 L as described in Table 1, and then transferred into tanks containing about 180 L of a specific culture medium for each microorganism. Table 2 shows a culture medium specific for Pseudomonas chlororaphis and Chromobacterium subtsugae and Table 3 shows a culture medium specific for Pseudomonas fluorescens, incubated for about 24 to about 168 hours. The air flow rate is preferably from about 1.0 to about 15.0 Nm³/h (=0.16-1.25 vvm).

In a preferred embodiment, the step of mixing the Pseudomonas and/or Chromobacterium species is carried out at a temperature of about 22° C. to about 38° C. The air flow rate is preferably about 1.0 Nm³/h and about 2.5 Nm³/h (=0.0085-0.021 vvm). The pressure is preferably from about 0.5 to about 1.2 kgf/cm³. Stirring is preferably from about 40 hz to about 45 hz.

The present invention further provides a method of controlling insect pests in plant crops comprising applying an agricultural composition according to the present invention to the crop or field of crops plants. A composition according to the present invention is effective against various target insect pests of high economic relevance and of difficult control, such as, for example: (i) corn leafhopper (Dalbulus maidis), which is responsible for persistently spreading two mollicutes: Spiroplasma kunkelii (disease known as pale stunt) and phytoplasma (red stunt), in addition to rayado fino virus (Waquil, 2004); (ii) the whitefly (Bemisia tabaci), a highly disseminated pest in bean crops, but which has also gained importance in vegetable crops, and (iii) the brown stink bug (Euschistus heros), which causes damage that reduces the quality and weight of soybean seeds (Correa-Ferreira and Azevedo, 2002). In addition to these, control of other orders of insect pests already widely reported, such as Lepidoptera and Coleoptera, can also be achieved by the present invention.

The present invention further provides a process of controlling diseases and crop pests of plants of agricultural interest comprising the application of a composition according to the present invention to agricultural crops.

The compositions and processes of the present invention are useful in the control of diseases, in particular two mollicutes, Spiroplasma kunkelii and phytoplasma, also known as pale stunt and red stunt, respectively. Both diseases are spread persistently by the maize leafhopper pest (Dalbus maidis).

The present invention further provides a process of controlling resistance of insect pests against agrochemicals in crops of plants 41 agronomic interest comprising applying an agricultural composition according to the present invention to the crop or field of plant crops.

In another embodiment, the present invention provides a method of promoting the growth of crop plants comprising applying an agricultural composition according to the present invention to the crop or field of crop plants.

Application of the composition according to the present invention is preferably made at doses ranging from about 0.5 to about 1.5 liters per hectare, and preferably with 1 to 3 applications during the crop cycle.

Plant crops of agronomic interest in accordance with the present invention include, but are not limited to, cotton, soybeans, corn, wheat, rice, among others.

Application of a composition according to the present invention can be carried out in different ways known to the person skilled in the art, for example, via foliar spray, via seed or sowing furrow. The following examples are only intended to illustrate one or more preferred embodiments of the invention and should not be construed as limitative of the scope of the invention.

### EXAMPLES

### Example 1—Culture Up Scale

The bacterial culture is inoculated in flasks containing 100 mL of culture medium as described in Table 1, being incubated in a 80-200 rpm orbital shaker at 22-38° C. for approximately 8 to 48 hours. The 100 mL inoculum are then transferred to flasks containing 1 L of culture medium, being incubated on an orbital shaker at 80-200 rpm at 22-38° C. for approximately 8-48 hours. Preferably, cultures of different Pseudomonas and/or Chromobacterium species are grown separately.

After the incubation period, bacterial cultures are inoculated into stainless steel flasks containing 10 L of specific culture medium for each microorganism and incubated for approximately 18 to 96 hours at an air flow rate of 0.25 to 1.5 Nm³/h (=0.41-2.5 vvm) and a temperature ranging from 22-38° C.

TABLE 1
Culture medium used to grow Pseudomonas and
Chromobacterium up to the 10 L scale and Pseudomonas chlororaphis
up to the 200 L scale.

| | Reagents | | |
|---|---|---|---|
| 01 | K₂HPO₄ | 0.1 to 1 | g |
| 02 | KH₂PO₄ | 0.1 to 1 | g |
| 03 | MgSO₄•7H₂O | 0.1 to 0.1 | g |
| 04 | NaCl | 0.05 to 0.3 | g |
| 05 | Yeast extract | 1 to 5 | g |
| 06 | (NH₄)₂SO₄ | 0.2 to 4 | g |
| 07 | 10% FeCl₃ solution | 0.05 to 1 | mL |
| 08 | 10% MnSO₄ solution | 0.05 to 1 | mL |
| 09 | glycerol | 5 to 20 | mL |
| 10 | KNO3 | 0.5 to 3 | g |

| | | |
|---|---|---|
| 11 | Water | q.s.p. 1 L |

q.s.p: sufficient quantity to

After this time has elapsed, each culture containing two stainless steel flasks with 10 L of culture medium is inoculated into a tank containing 180 L of the specific culture medium for each microorganism, the specific culture medium for Pseudomonas chlororaphis being shown in Table 1; the culture medium specific for Pseudomonas fluorescens and Chromobacterium being shown in Table 2 and incubated for about 24 to 168 hours at an air flow rate of from 3.0-10.0 Nm³/h (=0.25−0.83 vvm) and a temperature ranging from 22 to 38° C.

TABLE 2
Culture medium used to grow Pseudomonas fluorescens
in 180 L and 2000 L vessels, Pseudomonas chlororaphis and
Chromobacterium subtsugae in 2000 L fermenters.

| | Reagents | | |
|---|---|---|---|
| 01 | K₂HPO₄ | 0.5 to 5 | g |
| 02 | KH₂PO₄ | 0.5 to 5 | g |
| 03 | MgSO₄•7H₂O | 0.1 to 1 | g |
| 04 | NaCl | 0.01 to 0.1 | g |
| 05 | Yeast extract | 1 to 5 | g |
| 06 | glycine | 1 to 10 | g |
| 07 | Peptone | 1 to 5 | g |
| 08 | tryptophan | 0.3 to 3 | g |
| 09 | meat extract | 1 to 5 | g |
| 10 | glycerol | 5 to 25 | mL |
| 11 | chitosan | 0.1 to 1 | g |

| | | |
|---|---|---|
| 11 | Water | q.s.p. 1 L |

q.s.p: sufficient quantity to

After the time of incubation of P. fluorescens, P. chlororaphis and Chromobacterium subtsugae cultures, fermentation vessels containing the 2,000 L cultures are then inoculated into 2,000 L fermenters containing the specific media described in tables 1 and 2 and the incubation time is preferably 24 to 72 hours at a temperature of 22° C. to 38° C. Air flow rate is preferably 1.0 Nm³/h to 2.5 Nm³/h (=0.0085-0.021 vvm). Pressure is preferably of from 1.0 to 2.0 kgf/cm². Stirring is preferably from 40 hz to 45 hz.

### Example 2—Mixture of Pseudomonas and Chromobacterium in a Bioreactor

For the mixture of Pseudomonas e Chromobacterium species in a 6,000 L fermenter, preferably, the sterilization process uses 3,600 L of the formulation of Table 3, the sterilization process of the culture medium is carried out for approximately 60 to 120 minutes at a temperature of about 121° C. to approximately 130° C. Preferably, sterilization is performed at a pressure of about 1.0 to 2.0 Kgf/cm². After the sterilization and cooling period, the tank containing Pseudomonas chlororaphis is then inoculated into a 2,000 L fermenter and the incubation time is preferably from 24 to 72 hours at a temperature of 22° C. to 38° C. The air flow rate is preferably 1.0 Nm³/h to 2.5 Nm³/h (=0.0085-0.021 vvm). Pressure is preferably of from 1.0 to 2.0 kgf/cm³. Stirring is preferably from 40 hz to 45 hz.

Preferably, after the end of the Pseudomonas chlororaphis incubation time, the Pseudomonas fluorescens tank mix is then inoculated into the 6000 L fermenter containing the Pseudomonas chlororaphis and Pseudomonas fluorescens species. Preferably, the incubation time is preferably of from about 30 to about 120 minutes.

At the same time, in another fermenter, the sterilization process uses 1,800 L of the formulation of Table 3 in a 2,000 L fermenter, the sterilization process of the culture medium being carried out for about 60 to about 120 minutes, at a temperature of about 121° C. to about 130° C. Preferably, sterilization is carried out at a pressure of from about 1.0 to about 2.0 kgf/cm². After the sterilization and cooling period, the Chromobacterium-containing tank is then inoculated into a 2,000 L fermenter and the incubation time is preferably from 24 to 72 hours at a temperature of 22° C. to −38° C. The air flow rate is preferably 1.0 Nm³/h to 2.5 Nm³/h (=0.0085-0.021 vvm). Pressure is preferably of from 1.0 to 2.0 kgf/cm³. Stirring is preferably from 40 hz to 45 hz.

After incubation of the bacterial cultures of Pseudomonas and Chromobacterium species, between about 1, 200 L to about 2,400 L of Chromobacterium containing cultures are then inoculated into the about 6,000 L fermenter containing the Pseudomonas species culture in culture volumes ranging from about 6.00 L to about 4,800 L. Preferably, the mixing time of the microorganisms is from about 60 to about 120 minutes.

Alternatively, the up-scaling process of the cultures can be used for larger volumes, but always respecting the proportion of each microorganism, between about 60% to about 80% Pseudomonas and about 20% to about 40% Chromobacterium, preferably about 40% to about 60% (v/v) Pseudomonas chlororaphis, about 10% to about 20% (v/v) P. fluorescens, and about 20% to about 50% (v/v) Chromobacterium subtsugae.

### Example 3—Induction of Metabolites

Induction of metabolites comprises adding the inducing compounds glycine and tryptophan in the culture medium used in the up-scaling step. Glycine and tryptophan inducing compounds are used in the culture medium at the concentrations described in Table 2, preferably between about 1 g/L to about 10 g/L glycine and from about 0.3 g/L to about 3 g/L tryptophan. Still relative to the culture medium, the carbon: nitrogen (C: N) ratio is 5:3. The induction step is preferably carried out at a temperature of about 22° C. to about 38° C. for about 24 to about 72 hours. Preferably, the induction step is carried out using an air flow rate of from about 1.0 Nm³/h (0.25 vvm) to about 10.0 Nm³/h (=0.83 vvm), under a pressure of about 0.1 Kgf/cm² to 1.0 Kgf/cm² and stirring of, preferably, about 40 Hz to about 45 Hz.

### Example 4—Packaging and Final Formulation

Preferably, the product is presented in liquid form, being packaged in bag-in-box or gallon bags. For such a physical, liquid nature, the product can be presented in packages containing 1, 2, 3, 5, 10, 20 and 50 L. In an alternative embodiment, the product is presented in its solid, powder form. For this embodiment, the product packaging volume is packages containing 1, 2, 5 and 10 kg.

### Example 5—Additional Components

In some preferred embodiments, the composition of the invention is formulated in combination with additional compounds of interest. Additional of interest comprise, for example, neem essential oil (Azadirachta indica), lemongrass essential oil (Cymbopogon citratus), basil essential oil (Ocimum basilicum) and rubber tree latex (Hevea brasilensis).

The compositions according to the present invention can be formulated, preferably in combination with carriers, stabilizers and other agriculturally acceptable components known in the art, including surfactants, antifoaming agents, thickeners, acidulants, preservatives and wetting agents.

### Example 6. Enhancement of the Bioinsecticidal Effect Against Different Pests and Crops of Agronomic Importance

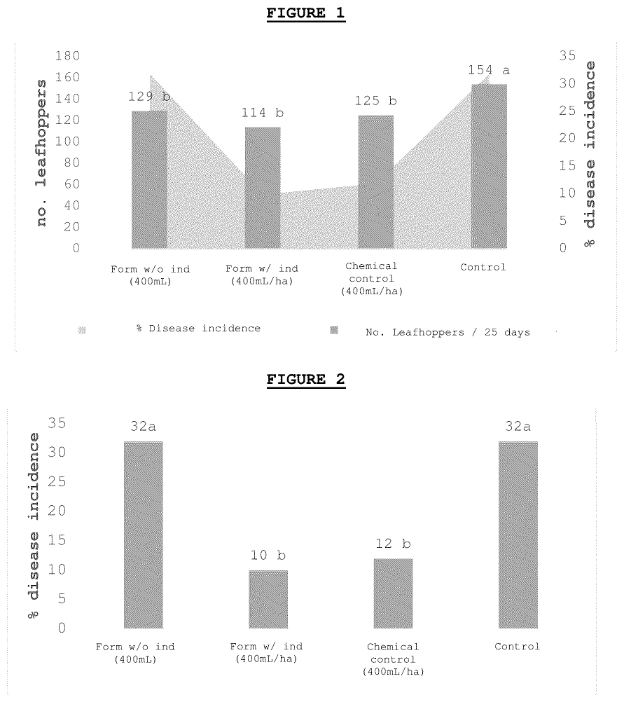

Field tests were carried out with different cultures and target insects in order to validate the effect of different formulations comprising Pseudomonas and/or Chromobacterium species and metabolites obtained by the process of inducing the same during the industrial process, as well as different dosages of the application aforementioned formulations. In the test carried out in a corn crop infected with Dalbulus maidis, also known as corn leafhopper (FIG. 1), the formulations with and without induction of metabolites from Pseudomonas species were shown to present a statistical difference as compared to the control, highlighting the formulation induced at doses of 400 and 600 mL/ha that showed better control of the target insect. In addition, all treatments with the bioinsecticide had the same effect as the chemical control used as a standard at a dosage as per the manufacturer's recommendations.

Corn leafhopper infestation has been causing increasingly important impacts, as in addition to causing direct damage to the crop, it is also capable of transmitting corn stunt viruses and mollicutes. Accordingly, the percentage of incidence of corn stunt disease was also assessed (FIG. 2), and similarly 400 and 600 mL/ha of the formulation with induction of metabolites provided a 75 and 69% reduction in the incidence of the disease, respectively. Massola et al (1999) observed that every 1% damage caused by the disease leads to up to 0.8% in corn yield, thus biological control can be a tool for controlling this pest in corn, since the bioinsecticide efficiency was equal to chemical control, with no statistical difference between treatments.

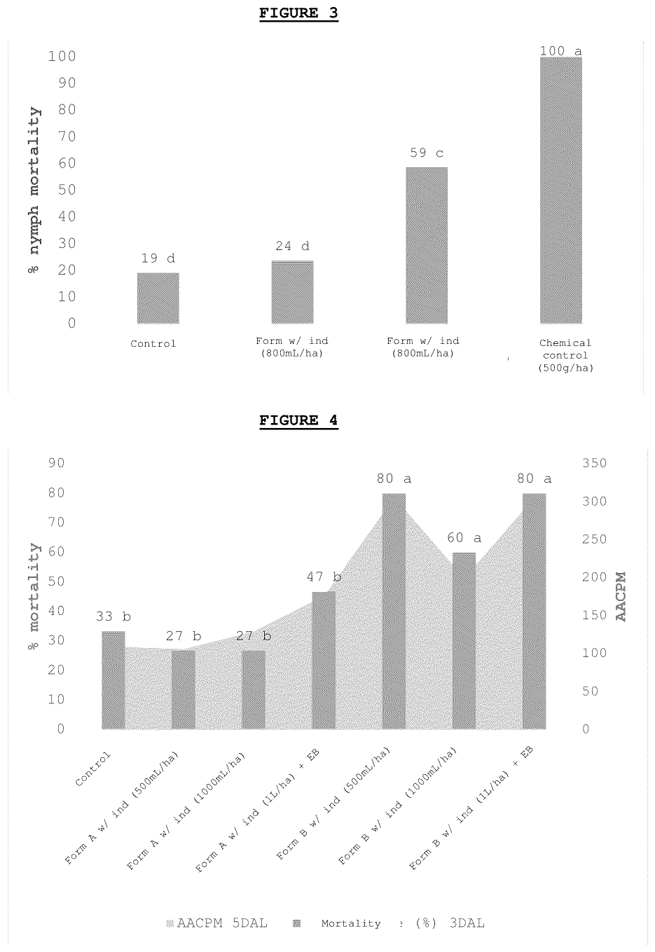

The whitefly (Bemisia tabaci), another pest of agricultural interest, is an insect that transmits the golden mosaic virus and dwarf mosaic virus, especially in bean crops, and acts mainly during the flowering period. In addition to the bean crop, the whitefly is also an important pest for soybean crops. Therefore, the effect of a composition according to the present invention comprising Pseudomonas P. fluorescens and P. chlororaphis, species on whitefly nymphs in bean crops was also evaluated (FIG. 3). All doses of the formulation with induction of metabolites and the highest doses of the formulation with no induction were shown to exhibit a statistical difference in the control of whitefly nymphs, where for the formulation with no induction of metabolites, 2 L/ha was required to achieve 250% mortality, while doses from 800 mL/ha of the formulation with induction of metabolites were sufficient to achieve the same control rate. In other words, the industrial process and the addition of compounds that induce bioactive metabolites against the whitefly, such as specific amino acids such as glycine and tryptophan, enhanced the insecticidal effect of the agricultural composition containing the species according to the present invention.

Also, with the aim of characterizing the spectrum of action of the agricultural composition disclosed herein, bioassays were carried out the brown stink bug (Euschistus heros), one of the most important soybean pests, using different Pseudomonas, P. fluorescens and P. chlororaphis and Chromobacterium subtsugae species as bioinsecticides (FIG. 4). In the bioassay, two formulations were assessed, which varied in the concentration of the glycine inducing compound (Table 2), formulation A comprising 1 g/L to 5 g/L glycine and formulation B comprising 6 g/L-10 g/L glycine. Thus, formulation B contains a greater amount of metabolites relative to formulation A, due to the addition of a higher concentration of the precursor compound of the metabolites of interest. As a result of this variation in concentration of the glycine inducing compound, the highest control rates of the brown stink bug were with formulation B, where application of the lowest dose tested was 500 mL/ha, showing 140% efficiency. The effect of the botanical extract containing azadirachtin compound associated with the intermediate dose of formulation B was also assessed. In this treatment, the extract was shown to increase the speed of action of the Bioinsecticide, and can be a good strategy to increase efficiency in the control of this insect, since increasing the microorganism dose has a negative effect on biocontrol.

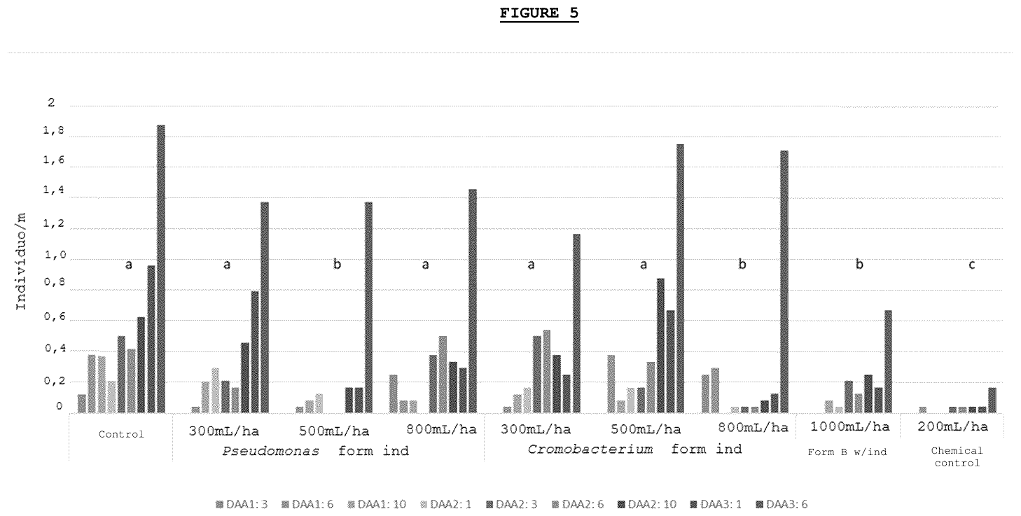

Although scientific reports of the individual insecticidal action of Chromobacterium subtsugae and Pseudomonas chlororaphis against pest insects of the order Hemiptera exist, no satisfactory positive effect was found in terms of insect control when the bacteria genera were applied alone in the field studies to assess brown stink bug nymphs and adults in soybean crops (FIG. 5). However, unexpectedly, control of this pest was shows to be more efficient when a composition comprising a mixture of Pseudomonas fluorescens and P. chlororaphis species together with Chromobacterium subtsugae was used.

Thus, it is evident that a composition according to the present invention comprising Pseudomonas fluorescens and P. chlororaphis associated with Chromobacterium subtsugae is an effective tool in the biological control of insects in different plant crops, in particular for target insects of agronomic interest that are difficult to manage.

### Example 7—Induction of Metabolites Also Promotes Plant Growth

Surprisingly, application of the agricultural composition according to the present invention to corn and soybean plants caused an additional effect related to the promotion of plant growth. The growth promotion achieved is of great importance, as it results in more robust and, accordingly, more productive plants. The results presented in table 3 show that a greater root length was achieved in all plants inoculated with microorganisms of Pseudomonas, P. fluorescens and P. chlororaphis species (Table 4), formulation 3 being highlighted. In the same way, superior results were evidenced for the parameter fresh mass from aerial part, with statistical differences as compared to the inoculated control. These results corroborate those obtained in soybean seedlings (Table 5), where once again formulation 3 showed greater root growth promotion as compared to the non-inoculated control.

In view of the results achieved, we investigated which mechanisms of action used by the bacteria would be linked to the observed promoting growth effect. The Pseudomonas species used as insecticidal agents were shown to be able to synthesize compounds related to the promotion of plant growth, namely indoleacetic acid (IAA) and siderophores. From the evidence of the presence of these compounds, quantitative methods were applied to assess the production of AIA and siderophores, the Salkowski colorimetric method (Glickmann and Dessaux, 1995) and the method described by Schwyn and Neilands (1987), respectively.

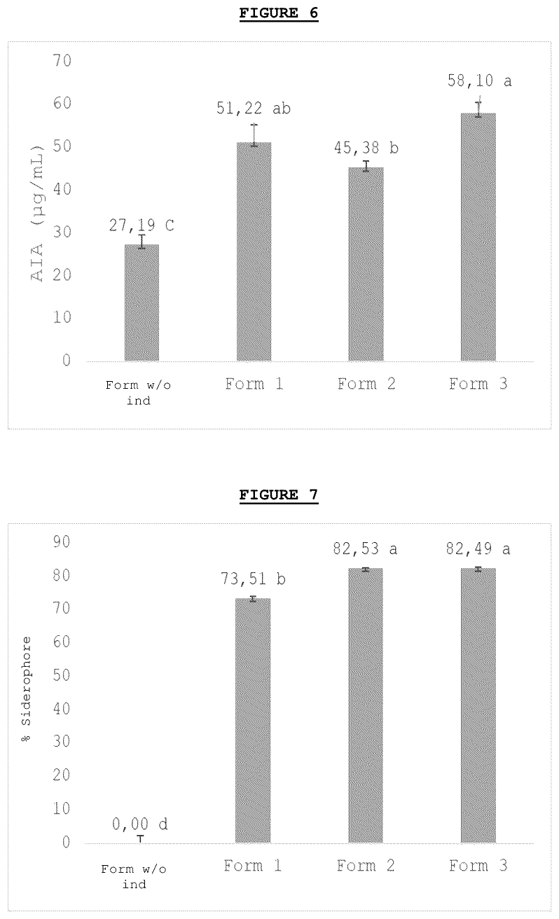

By applying the quantitative methods to characterize the different formulations tested, either induced or not, it was observed that the Pseudomonas species constitutively synthesize 27.19 μg/mL AIA and increase this amount by 113% when subjected to the industrial process and culture medium used to obtain the insecticidal agricultural composition (FIG. 6).

Production of siderophores was also quantified (Schwyn and Neilands 1987) (FIG. 7). Once again, the formulation and the industrial process applied to obtain the insecticidal agricultural composition provided high concentrations of siderophores, contrary to the formulation with no induction, which did not show any production of this compound.

In view of the results obtained, the industrial method to obtain the agricultural composition having insecticidal activity comprising different species of Pseudomonas, P. fluorescens and P. chlororaphis, in association with Chromobacterium subtsugae was shown to promote effective and surprising effects in promoting plant growth by providing AIA and Siderophore biosynthesis.

In addition to inducing bioinsecticidal metabolites in the formulations, another embodiment of the present invention was the induction of biosynthesis of indole acetic acid (IAA). As seen in FIG. 6, even without the addition of the tryptophan precursor, Pseudomonas species use tryptophan-independent pathways to produce AIA, since it was found that the formulation with no induction of Pseudomonas species synthesized 27.19 μg/mL AIA according to the Salkowski colorimetric process (Glickmann and Dessaux, 1995). However, when the precursor was added, the species produced up to 113% more AIA when compared to the formulation with no induction.

Production of siderophores was also quantified (Schwyn and Neilands 1987) in the differente formulations (FIG. 7). All of the induced formulations provided high siderophore concentrations, unlike the formulation with no induction, which did not show any production of this compound. Production of siderophores by microorganisms can promote plant that growth, is, either directly by facilitating the acquisition of iron (Fe), or indirectly, by inhibiting the establishment of pathogens by sequestering Fe from the environment, thus limiting the amount of this metal available for pathogen growth, hence providing better plant health (RADZKI et al., 2013). Association of the production of these two compounds was confirmed when growth promotion of corn and soybean plants was assessed. Table 3 shows a greater root length in all plants inoculated with Pseudomonas microorganisms, with emphasis on formulation 3 that has the lowest concentration of the formulation of metabolite-inducing molecules; these results on root growth are directly related to the higher concentration in AIA production by these microorganisms; similarly, the fresh mass from the aerial part (Table 4) also showed a statistical difference when compared to the inoculated control. These results corroborate those obtained in soybean seedlings (Table 5), where once again formulation 3 showed greater root growth promotion as compared to the non-inoculated control.

TABLE 3
Corn seed germination: root length up to 6 days after sowing
(DAS), root fresh mass (RFM) and root dry mass (RDM).

| | | 2 DAS | 3 DAS | 4 DAS | 5 DAS | 6 DAS | RFM | RDM |
|---|---|---|---|---|---|---|---|---|
| TREATMENTS | | (cm) | (cm) | (cm) | (cm) | (cm) | (g) | (g) |
| T1 | Control with no inoculation | 6.69 a | 30.91 b | 60.83 b | 84.31 b | 109.67 b | 0.191 a | 0.0224 ab |
| T2 | Form with no induction | 7.32 a | 40.88 a | 74.02 a | 99.68 a | 130.68 a | 0.163 a | 0.0220 ab |
| T3 | Form 1 | 7.05 a | 38.88 a | 66.92 ab | 95.38 ab | 122.27 a | 0.182 a | 0.0185 b |
| T4 | Form 2 | 6.69 a | 34.72 ab | 66.31 ab | 93.46 ab | 127.15 a | 0.156 a | 0.0218 ab |
| T5 | Form 3 | 9.81 a | 40.61 a | 72.79 a | 100.61 a | 132.58 a | 0.204 a | 0.0284 a |
| cv | (%) | 69.40 | 24.30 | 19.82 | 16.44 | 15.63 | 61.91 | 42.32 |

Means (4 triplicates) followed by the same letter in the same column showed no statistical difference between treatments by the Tukey's test (≤0.05).

TABLE 4
Corn seed germination: aerial part length 6 days after sowing (DAS),
aerial part fresh mass (APFM) and aerial part dry mass (APDM).

| | | 6 DAS | APFM | APDM |
|---|---|---|---|---|
| TREATMENTS | | (cm) | (g) | (g) |
| T1 | Control with | 34.45 ns | 0.124 b | 0.0146 ns |
| | no inoculation | | | |
| T2 | Form with no | 40.79 | 0.154 ab | 0.0160 |
| | induction | | | |
| T3 | Form 1 | 37.78 | 0.140 ab | 0.0148 |
| T4 | Form 2 | 40.75 | 0.140 ab | 0.0158 |
| T5 | Form 3 | 40.78 | 0.155 a | 0.0166 |
| cv | (%) | 21.34 | 24.96 | 24.81 |

Means (4 triplicates) followed by the same letter in the same column showed no statistical difference between treatments by the Tukey's test (≤0.05).

TABLE 5
Soybean seed germination: root length up to 6 days after sowing (DAS), root fresh mass (RFM) and root dry mass (RDM).

| | | 2 DAS | 3 DAS | 4 DAS | 5 DAS | 6 DAS | RFM | RDM |
|---|---|---|---|---|---|---|---|---|
| TREATMENTS | | (cm) | (cm) | (cm) | (cm) | (cm) | (g) | (g) |
| T1 | Control with no inoculation | 2.63 ns | 18.27 ns | 30.11 c | 54.66 ns | 70.25 b | 0.176 ns | 0.021 ns |
| T2 | Form with no induction | 1.85 | 16.79 | 31.95 bc | 60.23 | 86.37 ab | 0.169 | 0.018 |
| T3 | Form 1 | 2.85 | 20.25 | 38.87 ab | 67.95 | 91.12 ab | 0.189 | 0.021 |
| T4 | Form 2 | 4.73 | 21.25 | 36.99 abc | 66.96 | 85.05 ab | 0.179 | 0.021 |
| T5 | Form 3 | 5.51 | 23.73 | 40.45 a | 73.68 | 106.86 a | 0.211 | 0.023 |
| cv | (%) | 180.4 | 41.06 | 34.27 | 34.48 | 41.66 | 29.63 | 27.05 |

### REFERENCES

ANDERSON, A. J.; KIM, Y. C. Biopesticides produced by plant-probiotic Pseudomonas chlororaphis isolates. Crop Protection, v. 105, p. 62-69, 2018.
Boddey, R.; De Oliveira, O.; Urquiaga, S.; Reis, V.; De Olivares, F. et al. Biological nitrogen fixation associated with sugar cane and rice: contributions and prospects for improvement. Plant Soil. v. 174, n. 1-2, p. 195-209, 1995.
Bric, J. M.; Bostock, R. M.; Silverstone, S. E. Rapid in situ assay for indoleacetic acid production by bacteria immobilized on a nitrocellulose membrane. Appl Environ Microbiol. v. 57, n. 2, p. 535-538, 1991.
Brown, Peter H.; H O, Tuan-Hua David, Barley aleurone layers secrete a nuclease in response to gibberellic acid: purification and partial characterization of the associated ribonuclease, deoxyribonuclease, and 3′-nucleotidase activities, Plant Physiology, v. 82, n. 3, p. 801-806, 1986.
Cappucino, J. C.; Sherman, N. Nitrogen Cycle. In: Microbiology: A Laboratory Manual. (4th edn), Benjamin/Cumming Pub Co, New York, USA, p. 311-312, 1992.
Corrêa-Ferreira, B. S; Azevedo, J. Soybean seed damage by different species of stink bugs. Agric For Entomol v. 4, p. 145-150, 2002.
Daoust, R. A. Commercialization of bacterial insecticides. In: Proceedings and abstracts, Vth International Colloquium on Invertebrate Pathology and Microbial Control, Adelaide, Australia, 20-24 Aug. 1990. Department of Entomology, University of Adelaide, p. 7-11, 1990.
Dangar, R.; Tushar, Infectivity and ecology of K. Pseudomonas spp. from natural epizootics in the rice leaf folder, C naphalocrocis medinalis (Lepidoptera: Pyralidae) in India. Biocontrol Science and Technology, v. 18, n. 3, p. 241-253, 2008.
Duran, N.; Erazo, S.; Campos, V. 1983. Bacterial chemistry II. Antimicrobial photoproduct from pigment of Chromobacterium violaceum. An. Acad. Bras. Cién. 55:231-234.
Duran, N.; Antonio, Haun, M.; Pilli, R. A. Biosynthesis of a trypanocide by Chromobacterium violaceum. World J. Microbiol. Biotechnol. V. 10, p. 685-690, 1994.
Fasim, F.; Ahmed, N; Parsons, R.; Gadd, G. M. Solubilization of zinc salts by a bacterium isolated from the air environment of a tannery. FEMS Microbiol Lett. v. 213, n. 1, p. 1-6, 2002.
Flury, P.; Vesga, P.; Pechy-Tarr, M.; Aellen, N.; Dennert, F.; Hofer, N., Kupferschmied, K. P.; Kupferschmied, P.; Metla, Z.; Ma, Z.; Siegfried, S.; De Weert, S.; Bloemberg, G.; Hofte, M.; Keel, C. J.; Maurhofer, M. Antimicrobial and insecticidal: cyclic lipopeptides and hydrogen cyanide produced by plant-beneficial Pseudomonas strains CHA0, CMR12a, and PCL1391 contribute to insect killing. Front. Microbiol. v. 8, 100, 2017.
Glickmann E, Dessaux Y. A critical examination of the specificity of the Salkowski reagent for indolic compounds produced by phytopathogenic bacteria. Appl. Environ. 4 Microbiol. v. 61, n. 2, p. 793-796, 1995.
Habib, M. E. M.; Andrade, C. F. S. “Bactérias entomopatogênicas. In: Controle Microbiano de Insetos. Alves, S B (Coord.).”, 1986.
Hu X, Chen J, Guo J. Two Phosphate- and Potassium-solubilizing Bacteria Isolated from Tianmu Mountain, Zhejiang, China. World J Microbiol Biotechnol. v. 22, n. 9, p. 983-990, 2006.
Lee, J. H.; Ma, K. C.; Ko, S. J.; Kang, B. R; Kim, I. S; Kim, Y. C. Nematicidal Activity of a Nonpathogenic Biocontrol Bacterium Pseudomonas chlororaphis 06. Current Microbiology. v. 62, p. 746-751, 2011.
Jacobson, C. B.; Pasternak, J.; Glick, B. R. Partial purification and characterization of 1-aminocyclopropane-1-carboxylate deaminase from the plant growth promoting rhizobacterium Pseudomonas putida GR12-2. Can J Microbiol v. 40, n. 12, 1019-1025, 1994.
Kloepper, J.; Schroth, M. Plant growth-promoting rhizobacteria on radishes. In: Proceedings of the 4th international conference on plant pathogenic bacteria, p 879-882, 1978.
Kupferschmied, P.; Maurhofer, M.; Keel, C. Promise for plant pest control: root-associated pseudomonads with insecticidal activities. Frontiers in plant science, v. 4, p, 287, 2013.
Martin, P. A.w.; Hirose, E.; Aldrich, J. R. Toxicity of Chromobacterium subtsugae to southern green stink bug (Heteroptera: Pentatomidae) and corn rootworm (Coleoptera; Chrysomelidae), Journal of economic entomology, v, 100, n. 3, p. 680-684, 2007.
Massola, N. S.; Bedendo, I. P.; Amorin, L.; Lopes, J. R. S. Quantificação de danos causados pelo enfezamento vermelho e enfezamento pálido do milho em condições de campo. Fitopalogia BRasileira, v. 24, p. 136-142, 1999.
Pikovskaya, R. Mobilization of phosphorus in soil in connection with vital activity of some microbial species. Mikrobiologiya. v. 17, p. 362-370, 1948.
Radzki, W.; Gutierrez Mañero, F. J.; Algar, E.; Lucas García, J. A.; García-Villaraco, A.; Ramos Solano, B. Bacterial siderophores efficiently provide iron to iron-starved tomato plants in hydroponics culture. Antonie van Leeuwenhoek, V. 104, n. 3, p. 321-330, 2013.
Schippers, B., Bakker, A. W., Bakker, P. A. H. M., & Van Peer, R. Beneficial and deleterious effects of HCN-producing pseudomonads on rhizosphere interactions. Plant and Soil. v. 129 n. 1, p. 75-83, 1990.
Schwyn, B.; Neilands, J. Universal chemical assay for the detection and determination of siderophores. Anal Biochem v. 160, n. 1, p. 47-56, 1987.
Suganthi, M.; Senthilkumar, P.; Arvinth, S.; K N, C. Chitinase from Pseudomonas fluorescens and its insecticidal activity against Helopeltis theivora. The Journal of general and applied microbiology, 2016-11, 2007.
Suman, A.; Verma, P.; Yadav, N. A.; Saxena, A. K. Bioprospecting for extracellular hydrolytic enzymes from culturable thermotolerant bacteria isolated from Manikaran thermal springs. Res J Biotechnol. v. 10, p. 33-42, 2015.
Suman, A; Verma, P.; Yadav, A. N.; Srinivasamurthy, R.; Singh, A.; Prasanna, R. Development of hydrogel based bio-inoculant formulations and their impact on plant biometric parameters of wheat (Triticum aestivum L.). Int J Curr Microbiol Appl Sci. v. 5, n. 3, p. 890-901, 2016.
Tavares, W. S. et al. Potential use of Asteraceae extracts to control Spodoptera frugiperda (Lepidoptera: Noctuidae) and selectivity to their parasitoids Trichogramma pretiosum (Hymenoptera: Trichogrammatidae) and Telenomus remus (Hymenoptera: Scelionidae). Industrial Crops Products, v. 31, p. 384-388, 2009.
Waquil, J. M. Cigarrinha-do-milho: vetor de molicutes e vírus. Embrapa Milho e Sorgo-Circular Técnica (INFOTECA-E), 2004.
Yadav, A. N.; Verma, P.; Kumar, V.; Sachan, S. G.; Saxena, A. K. Extreme Cold Environments: A Suitable Niche for Selection of Novel Psychrotrophic Microbes for Biotechnological Applications. Adv Biotechnol Microbiol. v. 2, n. 2, p. 1-4, 2017.

1. Multi-target bioinsecticidal agricultural composition, said composition comprising at least one bacterium species of the genus Pseudomonas or the genus Chromobacterium, or a mixture thereof, wherein the composition is enriched in metabolites produced by said bacterium species.

2. The agricultural multi-target bioinsecticide composition according to claim 1, wherein said composition comprises a mixture of at least one bacterium species of the genus Pseudomonas, and one bacterium species of the genus Chromobacterium.

3. The multi-target bioinsecticidal agricultural composition according to claim 1 or 2, wherein the bacterium species of the genus Pseudomonas is selected from the group consisting of Pseudomonas chlororaphis and Pseudomonas fluorescens.

4. The multi-target bioinsecticidal agricultural composition according to any one of claims 1 to 3, wherein the bacterium species of the genus Chromobacterium is Chromobacterium subtsugae.

5. The multi-target bioinsecticidal agricultural composition according to any one of claims 1 to 4, wherein the ratio of each microorganism is from about 60% to about 80% Pseudomonas and from about 20% to about 40% Chromobacterium.

6. The multi-target bioinsecticidal agricultural composition according to claim 5, wherein the ratio of each microorganism is from about 40% to about 60% (v/v) Pseudomonas chlororaphis, from about 10% to about 20% (v/v) P. fluorescens from and from about 20% to about 50% (v/v) Chromobacterium subtsugae.

7. The multi-target bioinsecticidal agricultural composition according to any one of claims 1 to 6, wherein said composition comprises additional agriculturally acceptable components, preferably wherein the composition further comprises azadirachtin.

8. The multi-target bioinsecticidal agricultural composition according to any one of claims 1 to 7, wherein said composition is packaged in bags.

9. The process of manufacturing a multi-target bioinsecticidal agricultural composition, comprising the steps of:
(a) fermenting at least one bacterial culture comprising at least one bacterial species of the genus Pseudomonas or the genus Chromobacterium, or a mixture thereof, to obtain a fermented broth;
(b) inducing the production of metabolites by the bacteria present in the fermented broth upon cultivation in a medium comprising glycine and/or tryptophan; and
(c) formulating an agricultural composition comprising e fermented optionally, additional agriculturally acceptable components.

10. The process according to claim 9, wherein said method further comprises the sequential expansion of the bacterial culture for inoculation in the fermentation culture.

11. The process according to claim 10, wherein the sequential expansion is carried out at volumes of about 100 mL, about 1 L, about 10 L, about 180 L, about 2,000 L to about 6,000 L.

12. The process according to claim 10, wherein the bacterial culture is expanded by incubation in an orbital shaker at about 80 rpm to about 200 rpm.

13. The process according to claim 11, wherein the step of expanding the bacterial culture in a 10 L volume comprises incubation at an air flow rate of about 0.25 Nm³/h to about 1.0 Nm³/h (=0.41−1.67 vvm);

14. The process according to claim 13, wherein each step of sequential expansion comprises incubation for about 8 to about 48 horas.

15. The process according to any one of claims 9 to 14, wherein the culture fermentation is by batch.

16. The process according to claim 15, wherein the batch bacterial culture fermentation is made for about 24 to about 168 hours.

17. The process according to claim 13 or 14, wherein the bacterial culture fermentation is carried out at a pressure of about 1.0 to about 2.0 kgf/cm².

18. The process according to any one of claims 15 to 17, wherein the bacterial culture fermentation is carried out with stirring of about 40 hz to about 45 hz.

19. The process according to any one of claims 15 to 18, wherein the bacterial culture fermentation is carried out at a temperature of about 22° C. to about 38° C.

20. The process according to any one of claims 15 to 18, wherein the bacterial culture fermentation is carried out at an air flow rate of about 1.0 Nm³/h to about 2.5 Nm³/h (=0.0085−0.021 vvm).

21. The process according to any one of claims 15 to 18, wherein different bacterium species of the Pseudomonas and Chromobacterium genera are cultivated separately and mixed in a fermenter.

22. The process according to claim 21, wherein the Pseudomonas fermentation step is carried out in a volume of about 6,000 L.

23. The process according to claim 21, wherein the Chromobacterium fermentation step is carried out in a volume of about 2,000 L.

24. The process according to claim 23, wherein the mixture is carried out in a fermenter of about 6,000 L.

25. The process according to claim 24, wherein the mixture is carried out for about 60 to about 120 minutes.

26. The process according to any one of claims 9 to 25, wherein induction of the metabolite production takes place in a tank and fermenter environment.

27. The process, according to claim 26, wherein induction of the metabolite production takes place in a fermenter of about 2000 L.

28. The process according to any one of claims 9 to 27, wherein induction of the production of metabolites is carried out at a temperature of about 22° C. to 38° C.

29. The process according to any one of claims 9 to 28, wherein induction of the production of metabolites is carried out at an air flow rate of about 3.0 Nm³/h (0.25 vvm) to about 10.0 Nm³/h (=0.83 vvm).

30. A multi-target bioinsecticidal agricultural composition produced by the process as defined in claim 9.

31. A process for controlling insect pests of plants of agricultural interest, said process comprising applying the composition as defined in any one of claims 1 to 6 and 30.

32. A process for controlling plant diseases in plants of agricultural interest, said process comprising applying the composition as defined in any one of claims 1 to 6 and 30.

33. A process for controlling insect pest resistance to agrochemicals in plant cultures of plants of agricultural interest, said process comprising applying the composition as defined in any one of claims 1 to 6 and 30.

34. A method of promoting the growth of crop plants comprising applying an agricultural composition according to the present invention to the agricultural crop.

35. The process according to any one of claims 31 to 34, wherein the composition is applied via foliar spraying, seed or sowing furrow.

36. The process according to any one of claims 31 to 34, wherein the plants of agronomic interest are selected from the group consisting of cotton, soybean, corn, wheat and rice.

37. The process according to any one of claims 31 to 34, wherein application of said composition is made at doses ranging from about 0.5 to about 1.5 liters per hectare, with 1 to 3 applications during the culture cycle period.

38. The process according to claim 31 or 33, wherein the insect pest is selected from insects of the order Lepidoptera, Coleoptera and Hemiptera.

39. The process according to claim 38, wherein the insect pest is selected from the group consisting of corn leafhopper (Dalbulus maidis), whitefly (Bemisia tabaci) and brown stink bug (Euschistus heros).

40. The process according to claim 38, wherein the disease is selected from the group of pale stunt and red stunt.
